(19) United States
(12) Patent Application Publication
(10) Pub. No.: US 2009/0254105 A1
(43) Pub. Date: Oct. 8, 2009

(54) Anastomotic connectors

(75) Inventor: Dustin Thompson, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/062,709

(22) Filed: Apr. 4, 2008

(51) Int. Cl. A61B 17/115

(52) U.S. Cl. 606/153

(57) ABSTRACT

Methods and anastomotic connector device usable for connecting adjacently located blood vessels, ducts or other natural or artificial anatomical structures that have walls with openings formed therein. The anastomotic connector devices may be delivered through catheters and may be initially deployed in a non-collapsed configuration and subsequently transitioned to a collapsed configuration whereby they hold the adjacent vessels or structures in substantially abutting contact such that fluid or other matter may flow from one anatomical structure into the other.

### FIELD OF THE INVENTION

The present invention relates generally to medical devices and methods, and more particularly to devices and methods for creating anastomotic junctions between adjacent anatomical conduits, other hollow anatomical structures and/or natural or synthetic tubular grafts.

### BACKGROUND

In surgery, various types of anastomotic connections are frequently formed between luminal anatomical or hollow structures such as blood vessels, ducts, segments of intestine, etc. or for connecting tubular grafts of natural or synthetic material to a blood vessel or other anatomical structure.

In general, surgical anastomotic connections of anatomical conduits, such as blood vessels, fall into three categories; i.e., end-to-end, end-to-side, or side-to-side. Irrespective of which type of anastomotic connection is being formed, the usual surgical technique requires that the luminal anatomical conduit(s) be maneuvered into proximity and placed in abutting juxtaposition, such that the ends or openings of the anatomical conduit(s) are in alignment with one another. Thereafter, sutures, staples or other connecting apparatuses are passed through the walls of the juxtapositioned anatomical conduit(s) to form the desired anastomotic connection therebetween. Anastomotic connections of this type are frequently performed during surgical procedures wherein a diseased or injured segment of an anatomical conduit (e.g., blood vessel, intestine, etc.) has been resected and removed, and the opposing cut ends of the conduit are then reconnected (by end-to-end, side to side, or end to side anastomosis) to permit the desired flow of bodily fluids or other matter.

Modern medicine includes advanced catheter technologies that may be used to form side-to-side connections between blood vessels or other body conduits. For example, certain tissue penetrating catheter devices and methods are known for performing transluminal, catheter-based procedures wherein flow-through connections are formed between two adjacently situated anatomical conduits (e.g., blood vessels) to bypass a diseased, injured or obstructed segment of one of those anatomical conduits, using a segment of the adjacent conduit as the bypass loop. These procedures include Percutaneous Transluminal In Situ Coronary Venous Arterialization (PICVA) procedures wherein a tissue penetrating catheter is used to form openings in the walls of an obstructed coronary artery and an adjacent coronary vein and an anastomotic connector is implanted therebetween to connect the obstructed artery to the adjacent vein such that blood will flow from the obstructed artery into the adjacent coronary vein. An embolic blocker is then implanted in the coronary vein proximal to the anastomotic connection, causing the arterial blood to flow in the retrograde direction through the coronary vein, thereby providing needed perfusion of ischemic myocardium. This procedure is described in U.S. Pat. Nos. 6,746,464 (Makower), 6,669,709 (Cohen et al.), 6,685,716 (Flaherty et al.), 6,660,024 (Flaherty et al.), 6,579,311 (Makower), 6,561,998 (Roth et al.), 6,379,319 (Garibotto et al.), 6,375,615 (Flaherty et al.), 6,302,875 (Makower et al.), 6,283,983 (Makower et al.), 6,190,353 (Makower et al.), 6,159,225 (Makower), 6,068,638 (Makower) and 5,830,222 (Makower), the entire disclosure of each such patent being expressly incorporated herein by reference. The PICVA procedure is also described in: Osterle, Stephen N., et al., Percutaneous In Situ Coronary Venous Arterialization: Report of the First Human Catheter-Based Coronary Artery Bypass; Circulation; 103:2539-2543 (2001).

In another catheter-based procedure, known as a Percutaneous In Situ Coronary Artery Bypass (PICAB), the above-described steps of the PICVA procedure are performed initially. Thereafter, similar catheter-based techniques are used to create an additional anastomotic junction between the arteralized vein and the obstructed artery at a site downstream of the obstruction (or some other coronary artery), thereby allowing the arterial blood which had flowed into the coronary vein to reenter the obstructed artery (or some other coronary artery), after having bypassed the arterial obstruction. This PICAB procedure as well as other catheter-based devices and procedures for connecting blood vessels and other anatomical conduits are further described in U.S. Pat. Nos. 6,746,464 (Makower), 6,669,709 (Cohen et al.), 6,685,716 (Flaherty et al.), 6,660,024 (Flaherty et al.), 6,579,311 (Makower), 6,561,998 (Roth et al.), 6,379,319 (Garibotto et al.), 6,375,615 (Flaherty et al.), 6,302,875 (Makower et al.), 6,283,983 (Makower et al.), 6,190,353 (Makower et al.), 6,159,225 (Makower), 6,068,638 (Makower) and 5,830,222 (Makower), which are expressly incorporated herein by reference.

Also, U.S. Pat. No. 6,579,311 (Makower), which is also hereby expressly incorporated herein by reference, describes certain thoracoscopic or minimally invasive methods for by-passing an obstructed coronary artery by maneuvering, into juxtaposition with the obstructed artery, a tubular graft. Openings are formed in the juxtapositioned graft and in the adjacent artery, at sites upstream and downstream of the obstruction. The graft is then connected to the artery such that the openings in the graft are positioned in alignment with, and in fluidic connection with, the openings in the artery. Blood may then flow through the connections between the tube graft and the artery, thereby bypassing an obstructed region of the artery.

Certain catheter-deployable anastomotic connector devices useable for making vessel to vessel or graft to vessel connections have been devised. For example, U.S. Pat. No. 6,231,587 (Makower) describes connector apparatus deliverable through a delivery catheter to a position between openings formed in the walls of adjacent first and second vascular structures (e.g., vessels), such connector apparatus being initially delivered while in a compact configuration and thereafter deployable to an operative configuration wherein at least one projection on one end of the connector apparatus engages the first vascular structure and at least one projection on the other end of the connector apparatus engages the second vascular structure. Also, U.S. Pat. No. 6,287,317 (Makower et al.) describes methods and apparatus for passing attachment apparatus (e.g., connector devices, staples, etc.) or connector material (e.g., suture thread, wire, cord, filament, monofilament, etc.) into or through the wall of a luminal anatomical structure (e.g., a blood vessel or other anatomical conduit) for the purpose of; i) closing the lumen of the anatomical structure, ii) forming an anastomotic junction between separate anatomical structures (or between approximated segments of the same anatomical structure), and/or iii) attaching an article (e.g., an endoluminal, extraluminal or transluminal graft) or other apparatus to the wall of the anatomical structure. Also, U.S. Pat. No. 6,616,675 (Evard et al.) describes anastomotic connectors and apparatus for forming and/or maintaining connections between openings formed in anatomical structures, such as blood vessels. The apparatus is initially deployed in a first configuration which is sufficiently compact to be delivered through the lumen of a catheter or cannula. Thereafter, the device is expanded to a second configuration whereby it engages the anatomical structures and forms or maintains the desired connection between openings in the anatomical structures. Also, U.S. Pat. No. 6,432,127 (Kim et al.) describes anastomotic connector devices which are useable to maintain fluidic connection between, or approximation of, openings formed in adjacent natural or prosthetic anatomical conduits (or adjacent openings formed in a single anatomical conduits). These connector devices generally comprise a plurality of radially expandable annular members having one or more elongate strut members extending therebetween. Initially, the device is mountable on or within a delivery catheter while in a radially compact configuration. After the delivery catheter has been inserted into the body, the device is caused to transition to a radially expanded configuration whereby it becomes implanted within the body so as to maintain the desired fluidic connection between, or the desired approximation of, the anatomical conduit(s).

There remains a need for the design and development of new anastomotic connector devices and it is desirable that such connector devices be implantable by percutaneous catheter-based techniques in order to avoid the need for open surgical exposure of the affected anatomy.

### SUMMARY OF THE INVENTION

The present invention provides methods and devices for

In accordance with the invention, there is provided one embodiment of an anastomotic connector device and method usable for connecting adjacent anatomical structures having walls and openings therein, such device comprising (i) a first ovoid member having a first end and a second end; (ii) a second ovoid member having a first end and a second end; (iii) a first end member connecting the first end of the first ovoid member to the first end of the second ovoid member and (iv) a second end member connecting the second end of the first ovoid member to the second end of the second ovoid member. This device is positionable, while in a non-collapsed configuration, such that the first ovoid member is within one of the anatomical structures, the second ovoid member is within the other anatomical structure and the first and second end members extend through the openings formed in both anatomical structures. Thereafter, this device is transitionable to a collapsed configuration whereby the first and send end members bend so as to capture portions of the walls of the first and second anatomical structures between the first and second ends of the first and second ovoid members, thereby maintaining the first and second anatomical structures in attachment to one another such that body fluid may flow from one of the anatomical structure into the other anatomical structure.

Further in accordance with the invention, there is provided another embodiment of an anastomotic connector device and method usable for connecting adjacent anatomical structures having walls and openings therein, such device comprising (i) a first ovoid member having a mid-region, a first end and a second end and (ii) a second ovoid member having a mid region, a first end and a second end; (iii) wherein the first and second ovoid members are disposed in side-by-side juxtaposition with their mid-regions connected to one another. One end of this device is initially advanced through the openings formed in the walls of the anatomical structures while the device is in a non-collapsed configuration. Thereafter, the device is transitioned to a collapsed configuration wherein the first and second ends of the first ovoid member move toward one another and the first and second ends of the second ovoid member move toward one another, capturing portions of the walls of the first and second anatomical structures between the first and second ends of the first ovoid member and between the first and second ends of the second ovoid member and thereby maintaining the first and second anatomical structures in attachment to one another such that body fluid may flow from one of the anatomical structure into the other anatomical structure.

Still further in accordance with the invention, there is provided yet another embodiment of an anastomotic connector device and method usable for connecting adjacent anatomical structures having walls and openings therein, such device comprising (i) a first ovoid member having a mid-region, a first end and a second end and (ii) a second ovoid member having a mid region, a first end and a second end; (iii) wherein the first and second ovoid members are in side-by-side juxtaposition with their mid-regions connected to one another. This device is initially advanceable through the openings formed in the walls of the anatomical structures while in a non-collapsed configuration and subsequently transitionable to a collapsed configuration wherein the first end of the first ovoid member moves toward the first end of the second ovoid member and the second end of the first ovoid member moves toward the second end of the second ovoid member, capturing portions of the walls of the first and second anatomical structures between the first end of the first ovoid member and the first end of the second ovoid member and between the second end of the first ovoid member and the second end of the second ovoid member, thereby maintaining the first and second anatomical structures in attachment to one another such that body fluid may flow from one of the anatomical structure into the other anatomical structure.

Further aspects, elements, embodiments, objects and advantages of the present invention will be appreciated by those of skill in the relevant art upon reading the detailed description and examples set forth herebelow.

### BRIEF DESCRIPTION OF THE DRAWINGS

FIG. 1 is a perspective view of one embodiment of an anastomotic connector of the present invention.

FIG. 2A is a schematic representation showing the manner in which the anastomotic connector of FIG. 1 may be manufactured by cutting a segment from a cylindrical tube.

FIG. 2B is a schematic diagram showing the anastomotic connector of FIG. 1 loaded into a delivery catheter.

FIG. 2C is a side view of the anastomotic connector of FIG. 1.

FIG. 2D is a top view of the anastomotic connector of FIG. 1.

FIG. 2E is an end view of the anastomotic connector of FIG. 1.

FIG. 2F is an enlarged perspective view of the anastomotic connector of FIG. 1 in a non-collapsed configuration.

FIG. 2G is an enlarged perspective view of the anastomotic connector of FIG. 1 in a collapsed configuration.

FIG. 3 is a perspective view of another embodiment of an anastomotic connector of the present invention.

FIG. 4 is a perspective view of yet another embodiment of an anastomotic connector of the present invention.

FIGS. 5A through 5F show steps in a method for transluminal, catheter based formation of a side-to-side anastomosis between adjacent anatomical conduits in accordance with the present invention.

### DETAILED DESCRIPTION AND EXAMPLES

The following detailed description and the accompanying drawings to which it refers provide non-limiting, non-exhaustive examples of the invention and do not limit the scope of the claimed invention in any way.

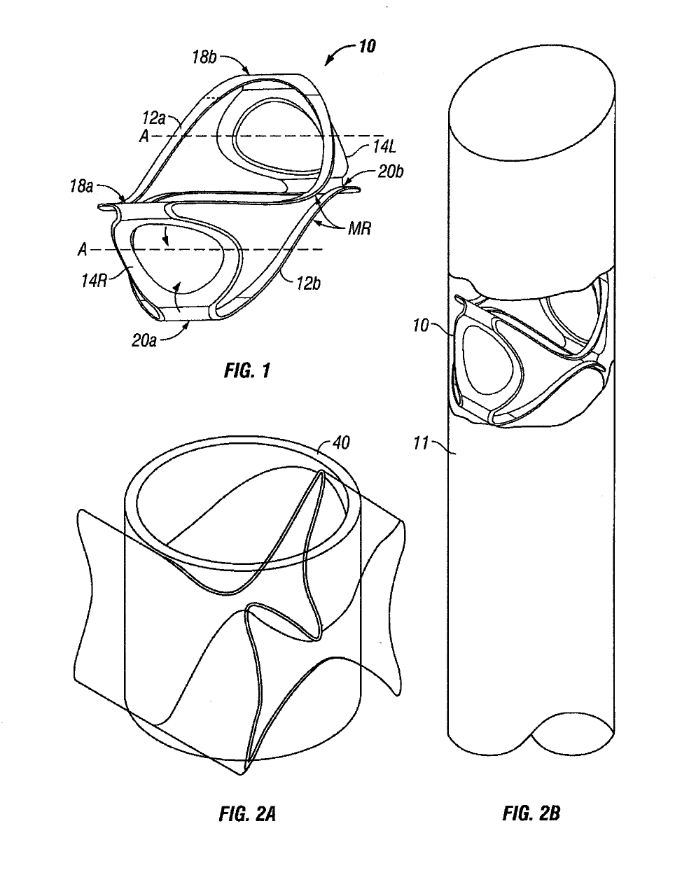

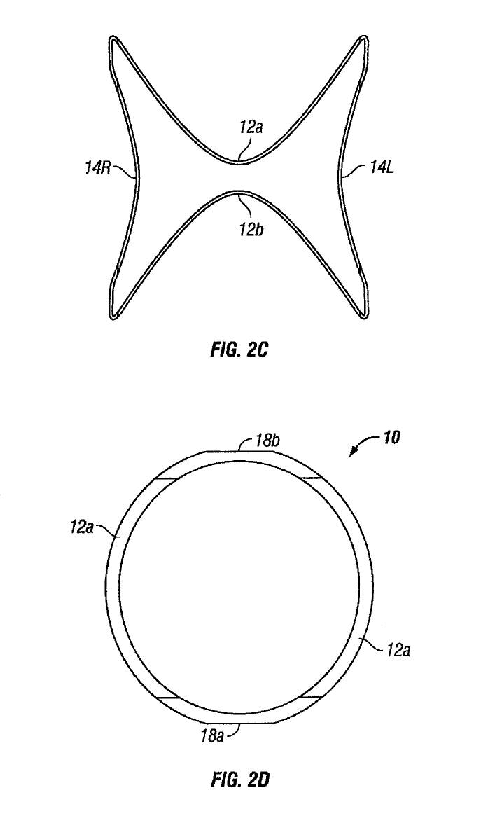

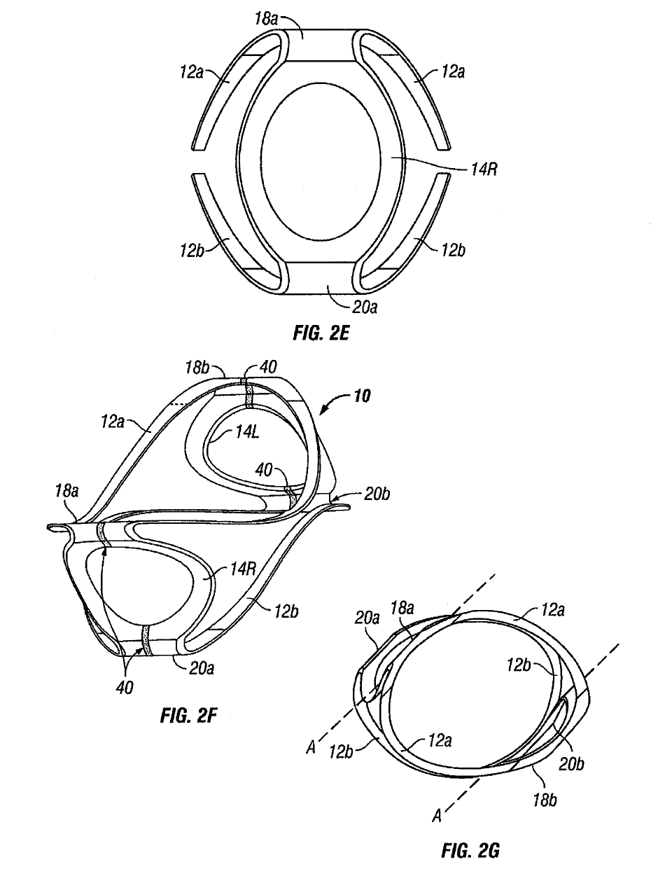

FIGS. 1 through 2G are directed to one embodiment of an anastomotic connector device 10 that is usable for connecting adjacent luminal or hollow anatomical structures such as blood vessels, ducts, segments of intestine, hollow organs, tubular or hollow grafts formed of natural or synthetic material, etc. In general, this device 10 comprises a first ovoid member 12a that has a first end 18a and a second end 18b, a second ovoid member 12b that has a first end 20a and a second end 20b, a first end member 14r that connects the first end 18a of the first ovoid member 12a to the first end 20a of the second ovoid member 12b and a second end member 141 that connects the second end 18b of the first ovoid member 12a to the second end 20b of the second ovoid member 12b. As seen in FIGS. 1 and 2F, the first and second ovoid members 12a, 12b may be curved such that their mid-regions MR are closer together than their ends 18a, 20a, 18b, 20b.

As will be described in more detail herebelow, this anastomotic connector device 10 is initially positionable, while in a non-collapsed configuration (seen in FIGS. 1 and 2F) such that the first ovoid member 12b is within one of the anatomical structures and the second ovoid member 12b is within the other anatomical structure and the first and second end members 14r, 14l extend through openings formed in the walls of both anatomical structures. Thereafter, the device 10 is transitionable to a collapsed configuration (shown in FIG. 2G) whereby the first and send end members 14r, 14l bend along each transverse axis A so as to capture adjacent portions of the walls of the first and second anatomical structures between the first ends 18a, 20a and second ends 18b, 20b, respectively, of the first and second end members 12a, 12b. This serves to hold the first and second anatomical structures in abutting approximation to one another in the area surrounding the openings formed therein such that body fluid may flow from one of the anatomical structure into the other anatomical structure.

As shown in FIG. 2, this anastomotic connector 10 may be easily manufactured by cutting (e.g., laser cutting) from a tubular workpiece 40 such as a segment of nitinol tube, stainless steel tube, cobalt chromium tube, platinum tube (materials common to self-expanding and balloon-expanding stents structures). Tubing diameters could range from, but are not limited to 1-4 mm with a wall thickness of 0.10 to 0.50-mm, depending upon the indicated use (e.g. the vessel sizes). Additionally, as shown in FIG. 2A, the required cut can be performed from one side of the tubular workpiece 40 such that rotation or manipulation of the tubular workpiece 40 will not be necessary during the cutting process.

After being cut from the tubular workpiece, the part is then worked to create the inward curvatures in the ovoid members 12a, 12b and end members 14r, 14l as seen in FIGS. 1 and 2F. For a self-collapsing embodiment of the device, following the cutting procedure, the cut-tube structure is incrementally deformed and heat treated until the final collapsed geometry is achieved (“Shape Memory Alloys” Sci. Am. November 1979, by L. McDonald Schetky, pp. 74-82). The super-elastic material properties of the self-collapsing design would allow it to be constrained within a delivery catheter 11 in the as-cut geometry (non-collapsed configuration) 2F, while returning to the heat-treated or collapsed configuration 2G upon delivery to a target location. For a plastically deformable geometry, the design would not require incremental deformation and heat-treatment, but would achieve its collapsed state by external forces (e.g. angioplasty type balloons) upon delivery to a target location.

This device may be loaded into a tubular delivery catheter 11 which may then be inserted into the subject's body and a push rod or other pushing member (not shown) may be used to advance the device 10 out of the open distal end of the delivery catheter 11. In self-collapsing embodiments, the device 10 may be elastic or superelastic and biased to its collapsed configuration. Such device 10 may be constrained in a non-collapsed configuration while inside the delivery catheter 11 and may then resiliently self-collapse as it advances out of the open distal end of the delivery catheter 11. In other embodiments, the device 10 may be plastically deformable mounted on an inner balloon catheter (not shown) that has 2 balloons, one ahead of and one behind the device 10. As the inner catheter is advanced out of the distal end of the delivery catheter 11, the balloons may be inflated, thereby compressing and plastically deforming the device 10 to its collapsed configuration.

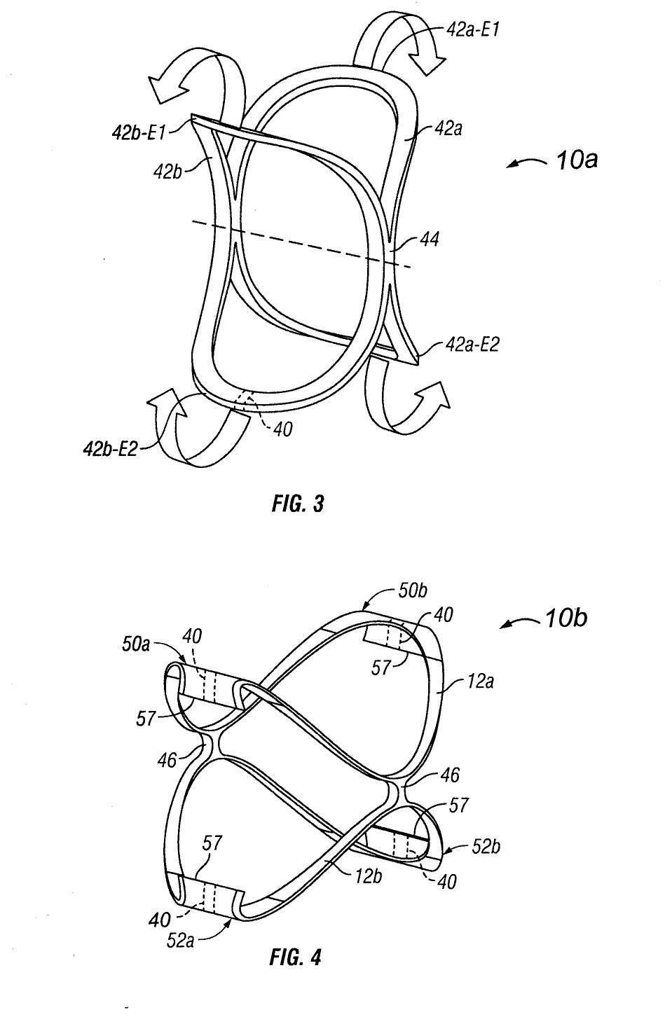

FIGS. 3 and 4 show alternative anastomotic connector devices 10a, 10b of this invention. The device 10a seen in FIG. 3 comprises a curved first ovoid member 42a and a curved second ovoid member 42b joined at their mid-regions by a solid connection 44. This device 10a is advanceable end first (e.g., ends 42a-e1 and 42a-e2 first) through openings that have been formed in the walls of adjacent anatomical structures. Thereafter, the ovoid members 42a, 42b are allowed to resiliently self-deform or are caused to plastically deform such that the ends 42a-e1 and 42a-e2 of the of the first ovoid member 42a move toward each other and the ends 42b-e1 and 42b-e2 of the second ovoid member 42b move toward each other. This is indicated by arrows on FIG. 3. This causes adjacent portions of the walls of the first and second anatomical structures to be captured between the juxtaposed ends 42a-e1 and 42a-e2 of the first ovoid member 42a and the juxtaposed ends 42b-e1 and 42b-e2 of the of the second ovoid member 42b, respectively. This serves to hold the first and second anatomical structures in abutting approximation to one another in the area surrounding the openings formed therein such that body fluid may flow from one of the anatomical structure into the other anatomical structure.

FIG. 4 shows another embodiment of an anastomotic connector device 10b which comprises first and second ovoid members 12a, 12b that are the same as those shown in the embodiment of FIG. 1. However, in this device 10a, the above-described side members 14r and 141 are absent and, instead, the ovoid members 12a, 12b are joined together at their mid-regions by solid connections 46. Also, in this device 10b, down-turned flanges 57 are formed on the ends 50a, 50b, 52a, 52b of the ovoid members 12a, 12b. This device 10b may be initially delivered through openings formed in first and second anatomical structures in a non-collapsed state (as shown in FIG. 4) in substantially the same manner as described above with respect to the device 10 of FIG. 1. Thereafter, the device 10b is transitioned to a collapsed stated whereby ovoid members 12a, 12b resiliently self-deform or are plastically deformed such that the ends 50a and 52a of the of the first and second ovoid members 12a, 12b close toward each other and the other ends 50b and 52b of the of the first and second ovoid members 12a, 12b also close toward each other. This captures adjacent portions of the walls of the first and second anatomical structures between ends 50a and 52a as well as between ends 50b and 52b. The down-turned flanges 57 may be configured to bite or penetrate into the tissue, thereby adding to the firmness of the implantation of this device 10b. This serves to hold the first and second anatomical structures in abutting approximation to one another in the area surrounding the openings formed therein such that body fluid may flow from one of the anatomical structure into the other anatomical structure. It is to be appreciated that, instead of flanges 57, one or more serrations, roughened area, spikes, teeth or other projection(s) may be formed on the ends 50a, 50b, 52a, 52b to aid in engaging or griping the underlying tissues.

Optionally, as shown, in some embodiments of these anastomotic connector devices 10, 10a, 10b, one or more radiopaque markers 40 (e.g., wire or foil that is radiographically distinguishable from the remainder of the device) may be may be placed at desired locations on the device 10 to assist the operator in positioning and deploying the device.

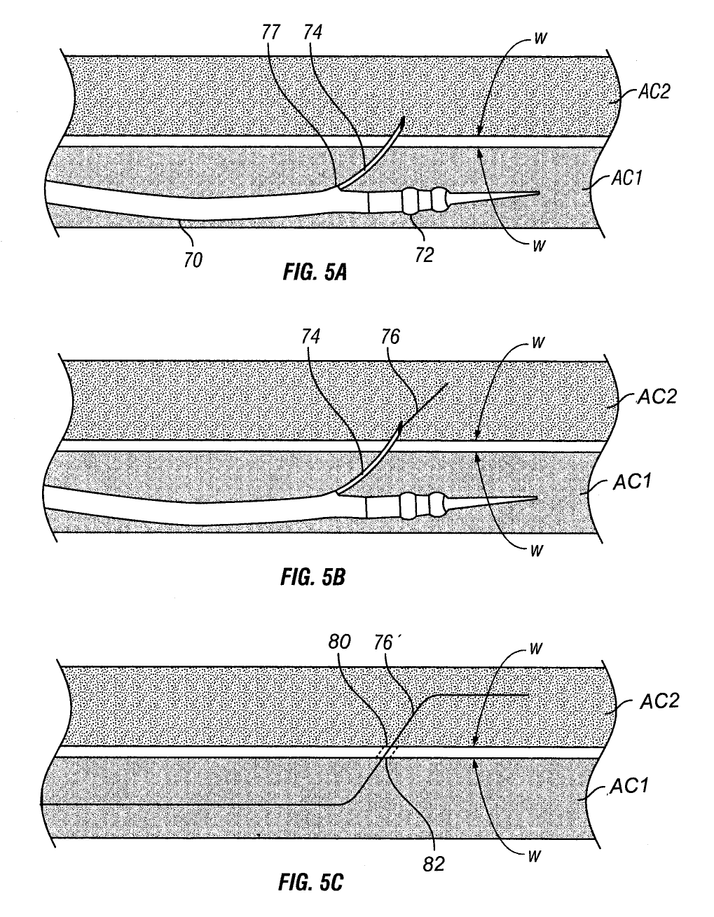

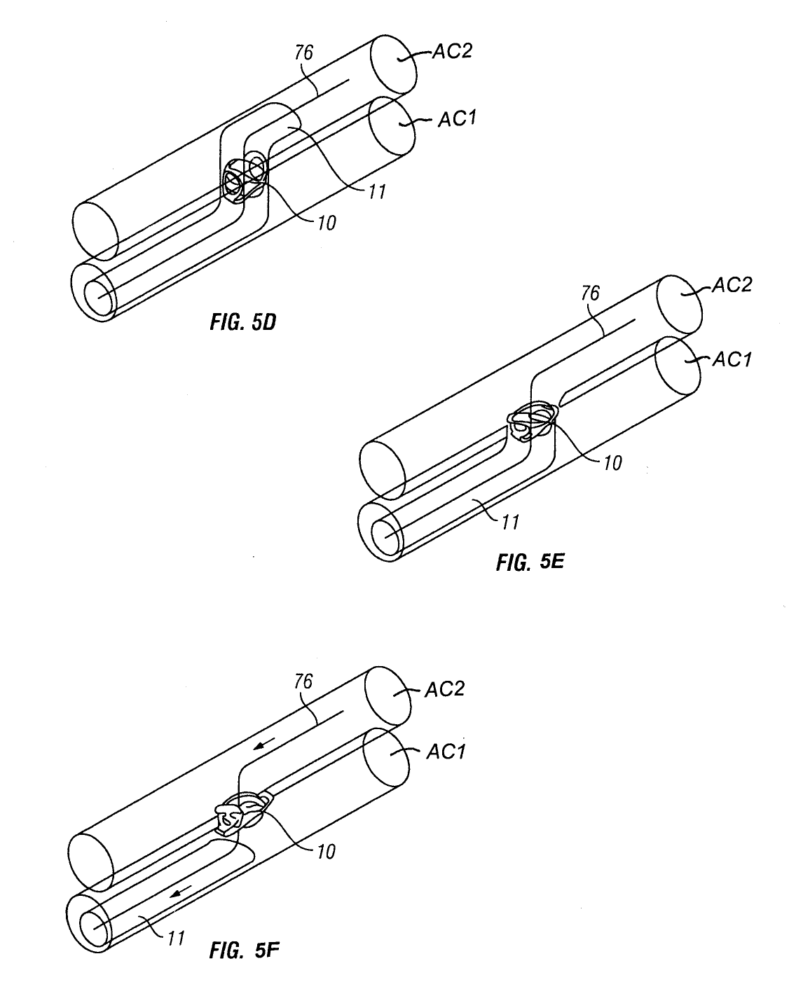

FIGS. 5A-5F show one example of a percutaneous, catheter-based method by which the connector device 10 shown in FIGS. 1-2G above may be implanted in openings formed in the walls W of adjacent anatomical conduits AC1, AC2. In this example, a tissue penetrating catheter 70 (Pioneer Catheter, Medtronic Vascular, Inc., Santa Rosa, Calif.) is advanced into the lumen of the first anatomical conduit AC1 and an on-board ultrasound imaging/orientation system 72 is used to locate the adjacent second anatomical conduit AC2 and to rotationally orient the catheter 70 within the first anatomical conduit AC1 such that the outlet port 77 of the tissue penetrating catheter 70 is properly aimed toward the second anatomical conduit AC2 so that, when the tissue penetrator 74 is subsequently advanced, it will travel in the direction of the second anatomical conduit AC2. Thereafter, the tissue penetrator 74 (which comprises a hollow needle) is advanced out of outlet port 77, through the wall W of the first anatomical conduit AC1 creating an opening 82 therein, through any intervening tissue located between the anatomical conduits, through the wall W of the second anatomical conduit AC2 creating an opening 80 therein and into the lumen of the second anatomical conduit AC2, as shown in FIG. 5A.

Thereafter, as seen in FIG. 5B, a guidewire 76 is advanced through the lumen of the penetrator 74 and into the lumen of the second anatomical conduit AC2.

Thereafter, as seen in FIG. 5C, the penetrator 74 is retracted into the tissue penetrating catheter 70 and the tissue penetrating catheter 70 is removed, leaving the guidewire 76 in place. Optionally, at this stage, a channel enlarging apparatus (not shown) such as a balloon catheter, laser, tissue cutting device, radiofrequency device, etc. may be advanced over the guidewire 76 to enlarge the openings 80, 82 formed in the walls of the anatomical conduits AC1, AC2 as well as any intervening tissue located between the anatomical conduits, if so desired. A detailed explanation of such channel enlarging procedure and further examples of channel enlarging devices that may be used for such channel enlarging procedure are described in U.S. Pat. No. 6,516,998 (Roth et al.), the entire disclosure of which is expressly incorporated herein by reference.

After any optional channel enlargement has been accomplished, the delivery catheter 11 having the anastomotic connector device 10 loaded therein is advanced over the guidewire 76 through the lumen of the first anatomical conduit AC1, through the opening 82 in the wall W of the first anatomical conduit AC1, through opening 80 formed in the wall of the second anatomical conduit AC2 and into the lumen of the second anatomical conduit AC2 such that the device 10 is positioned within the openings 80, 82 formed in the walls of the anatomical conduits AC1, AC2, as shown in FIG. 5D. Thereafter, a push rod (not shown) is positioned behind the anastomotic connector device 10 and the catheter 11 is retracted, causing the device to be expelled out of the distal end of the delivery catheter 11, as seen in FIG. 5E. In this example, the device 10 is a self-collapsing device which automatically assumes its collapsed configuration (seen in FIG. 2G) as it is expelled out of the delivery catheter 11. In this manner, the device 10 firmly clips onto tissue of the walls W of the anatomical conduits AC1, AC2 adjacent to the openings 80, 82, thereby holding the openings 80 and 82 in alignment with one another and preventing substantial leakage of fluid from the area between the openings 80 and 82.

After the device 10 has been implanted, the delivery catheter 11 and guidewire 76 are removed, as indicated by arrows on FIG. 5F. The device 10 remains implanted, thereby creating the desired side-to side anastomotic connection between the first anatomical conduit AC1 and the second anatomical conduit AC2.

It is to be further appreciated that the invention has been described here above with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless to do so would render the embodiment or example unsuitable for its intended use. Also, where the steps of a method or process are described, listed or claimed in a particular order, such steps may be performed in any other order unless to do so would render the embodiment or example not novel, obvious to a person of ordinary skill in the relevant art or unsuitable for its intended use. All reasonable additions, deletions, modifications and alterations are to be considered equivalents of the described examples and embodiments and are to be included within the scope of the following claims.

What is claimed is:

1. An anastomotic connector device usable for connecting adjacent anatomical structures having walls and openings therein, said device comprising:
a first ovoid member having a first end and a second end;
a second ovoid member having a first end and a second end;
a first end member connecting the first end of the first ovoid member to the first end of the second ovoid member;
a second end member connecting the second end of the first ovoid member to the second end of the second ovoid member;
said device being positionable, while in a non-collapsed configuration, such that the first ovoid member is within one of the anatomical structures, the second ovoid member is within the other anatomical structure and the first and second end members extend through the openings formed in both anatomical structures;
said device being thereafter transitionable to a collapsed configuration whereby the first and send end members bend so as to capture portions of the walls of the first and second anatomical structures between the first and second ends of the first and second ovoid members, thereby maintaining the first and second anatomical structures in attachment to one another such that body fluid may flow from one of the anatomical structure into the other anatomical structure.

2. A device according to claim 1 wherein the device is formed of elastic or superelastic material and is biased to the collapsed configuration such that it will self-contact to the collapsed configuration when unconstrained and at body temperature.

3. A device according to claim 1 wherein the device is initially disposed in the non-collapsed configuration and is plastically deformable to the collapsed configuration.

4. A device according to claim 1 further comprising one or more radiopaque markers.

5. A method for creating an anastomosis between first and second anatomical structures having walls and openings formed therein, said method comprising the steps of:
(A) providing an anastomotic connector device that comprises (i) a first ovoid member having a first end and a second end; (ii) a second ovoid member having a first end and a second end; (iii) a first end member connecting the first end of the first ovoid member to the first end of the second ovoid member and (iv) a second end member connecting the second end of the first ovoid member to the second end of the second ovoid member, said device being initially deployable in a non-collapsed configuration and subsequently transitionable to a collapsed configuration;
(B) advancing said device, while in the non-collapsed configuration, such that the first ovoid member is within one of the anatomical structures, the second ovoid member is within the other anatomical structure and the first and second end members extend through the openings formed in both anatomical structures;
(C) causing the device to transition to the collapsed configuration such that portions of the walls of the first and second anatomical structures are captured between the first and second ends of the first and second ovoid members, thereby maintaining the first and second anatomical structures in attachment to one another such that body fluid may flow from one of the anatomical structure into the other anatomical structure.

6. A method according to claim 5 wherein the device is self collapsing and wherein Step C comprises removing constraint from the device to allow it to self collapse to the collapsed configuration.

7. A method according to claim 5 wherein the device is plastically deformable and wherein Step C comprises exerting pressure on the device to plastically deform the device from the non-collapsed configuration to the collapsed configuration.

8. An anastomotic connector device usable for connecting adjacent anatomical structures having walls and openings therein, said device comprising:
a first ovoid member having a mid-region, a first end and a second end;
a second ovoid member having a mid region, a first end and a second end;
the first and second ovoid members being in side-by-side juxtaposition with their mid-regions connected to one another;
one end of said device being initially advanceable through the openings formed in the walls of the anatomical structures while in a non-collapsed configuration and subsequently transitionable to a collapsed configuration wherein the first and second ends of the first ovoid member move toward one another and the first and second ends of the second ovoid member move toward one another, thereby capturing portions of the walls of the first and second anatomical structures between the first and second ends of the first ovoid member and between the first and second ends of the second ovoid member, thereby maintaining the first and second anatomical structures in attachment to one another such that body fluid may flow from one of the anatomical structure into the other anatomical structure.

9. A device according to claim 8 wherein the device is formed of elastic or superelastic material and is biased to the collapsed configuration such that it will self-contact to the collapsed configuration when unconstrained and at body temperature.

10. A device according to claim 8 wherein the device is initially disposed in the non-collapsed configuration and is plastically deformable to the collapsed configuration.

11. A device according to claim 8 further comprising one or more radiopaque markers.

12. A method for creating an anastomosis between first and second anatomical structures having walls and openings formed therein, said method comprising the steps of:
(A) providing an anastomotic connector device that comprises (i) a first ovoid member having a mid-region, a first end and a second end and (ii) a second ovoid member having a mid region, a first end and a second end; (iii) wherein the first and second ovoid members are in side-by-side juxtaposition with their mid-regions connected to one another;
(B) advancing said device, while in a non-collapsed configuration, such that one end of said device passes through the openings formed in the walls of the anatomical structures; and
(C) causing the device to transition to a collapsed configuration wherein the first and second ends of the first ovoid member move toward one another and the first and second ends of the second ovoid member move toward one another, capturing portions of the walls of the first and second anatomical structures between the first and second ends of the first ovoid member and between the first and second ends of the second ovoid member and thereby maintaining the first and second anatomical structures in attachment to one another such that body fluid may flow from one of the anatomical structures into the other anatomical structure.

13. A method according to claim 12 wherein the device is self collapsing and wherein Step C comprises removing constraint from the device to allow it to self collapse to the collapsed configuration.

14. A method according to claim 12 wherein the device is plastically deformable and wherein Step C comprises exerting pressure on the device to plastically deform the device from the non-collapsed configuration to the collapsed configuration.

15. An anastomotic connector device usable for connecting adjacent anatomical structures having walls and openings therein, said device comprising:
a first ovoid member having a mid-region, a first end and a second end;
a second ovoid member having a mid region, a first end and a second end;
the first and second ovoid members being in side-by-side juxtaposition with their mid-regions connected to one another;
said device being initially advanceable through the openings formed in the walls of the anatomical structures while in a non-collapsed configuration and subsequently transitionable to a collapsed configuration wherein the first end of the first ovoid member moves toward the first end of the second ovoid member and the second end of the first ovoid member moves toward the second end of the second ovoid member, capturing portions of the walls of the first and second anatomical structures between the first end of the first ovoid member and the first end of the second ovoid member and between the second end of the first ovoid member and the second end of the second ovoid member, thereby maintaining the first and second anatomical structures in attachment to one another such that body fluid may flow from one of the anatomical structure into the other anatomical structure.

16. A device according to claim 15 wherein the device is formed of elastic or superelastic material and is biased to the collapsed configuration such that it will self-contact to the collapsed configuration when unconstrained and at body temperature.

17. A device according to claim 15 wherein the device is initially disposed in the non-collapsed configuration and is plastically deformable to the collapsed configuration.

18. A device according to claim 15 further comprising one or more radiopaque markers.

19. A device according to claim 15 further comprising flanges, projections or surface modifications which frictionally engage or penetrate into tissue that is captured between the first end of the first ovoid member and the first end of the second ovoid member and between the second end of the first ovoid member and the second end of the second ovoid member.

20. A method for creating an anastomosis between first and second anatomical structures having walls and openings formed therein, said method comprising the steps of:
(A) providing an anastomotic connector device that comprises (i) a first ovoid member having a mid-region, a first end and a second end and (ii) a second ovoid member having a mid region, a first end and a second end; (iii) wherein the first and second ovoid members are in side-by-side juxtaposition with their mid-regions connected to one another;
(B) advancing said device, while in a non-collapsed configuration, such that the first and second ends of one of the ovoid members passes through the openings formed in the walls of the anatomical structures; and
(C) causing the device to transition to a collapsed configuration wherein the first end of the first ovoid member moves toward the first end of the second ovoid member and the second end of the first ovoid member moves toward the second end of the second ovoid member, capturing portions of the walls of the first and second anatomical structures between the first end of the first ovoid member and the first end of the second ovoid member and between the second end of the first ovoid member and the second end of the second ovoid member, thereby maintaining the first and second anatomical structures in attachment to one another such that body fluid may flow from one of the anatomical structures into the other anatomical structure.

21. A method according to claim 20 wherein the device is self collapsing and wherein Step C comprises removing constraint from the device to allow it to self collapse to the collapsed configuration.

22. A method according to claim 20 wherein the device is plastically deformable and wherein Step C comprises exerting pressure on the device to plastically deform the device from the non-collapsed configuration to the collapsed configuration.
